(19) United States
(12) Patent Application Publication
(10) Pub. No.: US 2024/0389908 A1
(43) Pub. Date: Nov. 28, 2024

(54) ANALYTE MONITORING DEVICE CALIBRATION

(71) Applicant: Dexcom, Inc., San Diego, CA (US)

(72) Inventors: Nicholas Vincent Apollo, San Diego, CA (US); Jonathon Clemens, San Diego, CA (US); Wenjie Lan, Westford, MA (US); Chris Dring, San Diego, CA (US)

(21) Appl. No.: 18/673,130

(22) Filed: May 23, 2024

Related U.S. Application Data
(60) Provisional application No. 63/468,669, filed on May 24, 2023.

(51) Int. Cl. A61B 5/1495; A61B 5/145; A61B 5/1486

(52) U.S. Cl. CPC A61B 5/1495; A61B 5/145; A61B 5/14865

(57) ABSTRACT

Analyte monitoring device calibration is described. A calibration system generates calibration results for multiple analyte monitoring devices during a calibration evaluation process. To do so, the calibration system implements a calibration board that includes a single ground electrode shared by, but not physically connected to, the multiple analyte monitoring devices. An input voltage is applied to an analyte-sensing element of each of the multiple analyte monitoring devices. Analyte-sensing elements and the common ground electrode are placed into/in contact with a reference bath that includes a known concentration of an analyte to be measured. The input voltage causes each analyte-sensing element to generate an electrical current that is indicative of the reference bath analyte concentration. The calibration system includes a current meter that monitors the electrical current generated by each of the multiple analyte monitoring devices, which is interpreted by the calibration system to generate the calibration results.

### CROSS-REFERENCE TO RELATED APPLICATIONS

This application claims priority to and benefit of U.S. Provisional Patent Application No. 63/468,669, filed May 24, 2023, which is assigned to the assignee hereof and hereby expressly incorporated by reference herein in its entirety as if fully set forth below and for all applicable purposes.

### BACKGROUND

Metabolic conditions such as diabetes affect hundreds of millions of people. For these people, monitoring analyte levels (e.g., blood glucose levels) and regulating those levels to be within an acceptable range is important not only to mitigate long-term issues such as heart disease and vision loss, but also to avoid the effects of hyperglycemia and hypoglycemia. Maintaining blood glucose levels within an acceptable range is often difficult, as this level is almost constantly changing over time and in response to events and activities, such as medications, therapeutic treatments, eating, exercising, sleeping, stress, and so forth. For caregivers and medical providers tasked with caring for multiple patients simultaneously, monitoring and appropriately reacting to changing analyte levels is challenging. When using a device to monitor analyte levels, accurate monitoring critically depends on proper and accurate operation of the device.

### SUMMARY

To overcome these problems, systems and techniques for calibrating analyte monitoring devices are described. In implementations, a calibration system is configured to simultaneously generate calibration results for multiple analyte monitoring devices. The calibration results are representative of measurements generated by respective sensors of the multiple analyte monitoring devices during a calibration evaluation process. The calibration system implements a calibration board that includes a common ground electrode that is shared by the multiple analyte monitoring devices during the calibration evaluation process. Advantageously, the common ground electrode is not physically connected to the multiple analyte monitoring devices during the calibration evaluation process. The calibration system includes a voltage source that is configured to apply an input voltage to each of the multiple analyte monitoring devices via respective electrodes on the calibration board that are each connected to a sensor wire of a different one of the multiple analyte monitoring devices.

During the calibration evaluation process, sensor wires of the multiple analyte monitoring devices and the common ground electrode are placed into a reference bath that contains a known concentration of an analyte to be measured by the multiple analyte monitoring devices. The input voltage causes each sensor wire to generate an electrical current that is indicative of the concentration within the reference bath. The calibration system includes a current meter that monitors the electrical current generated by each of the multiple analyte monitoring devices, which the calibration system uses to generate calibration results for each of the multiple analyte monitoring devices. Analyte monitoring devices having satisfactory calibration results can be approved for use (e.g., by a patient and/or for determining calibration information for other sensor(s) that may be used by a patient). Analyte monitoring devices having unsatisfactory calibration results may be discarded and/or not used for determining calibration information for other sensor(s).

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 illustrates an environment in an example implementation that includes a calibration system operable to calibrate analyte monitoring devices in accordance with techniques described herein.

FIG. 2 depicts an example implementation of an analyte monitoring device calibrated in accordance with the techniques described herein.

FIG. 3 depicts an example of the calibration system of FIG. 1 in greater detail.

FIG. 4 depicts an example of an apparatus useable to calibrate analyte monitoring devices.

FIG. 5 depicts an example of an apparatus used by the calibration system of FIG. 1 to calibrate analyte monitoring devices.

FIG. 6 depicts a procedure in an example implementation of calibrating analyte monitoring devices using the calibration system of FIG. 1.

FIG. 7 illustrates an example of a system including various components of an example device that can be implemented as any type of computing device as described and/or utilized with reference to FIGS. 1-6 to implement the techniques described herein.

### DETAILED DESCRIPTION

### Overview

Proper calibration of analyte monitoring devices, such as glucose monitors, is critical for individuals who rely on these devices to monitor their analyte levels (e.g., blood glucose levels). Many analyte monitoring devices use sensors to measure analyte levels in bodily fluids (e.g., interstitial fluids, bloodstream etc.). Therefore, sensor calibration is paramount in ensuring that an analyte monitoring device provides accurate and reliable readings. When an analyte monitoring device is not calibrated properly, it can lead to serious consequences. For instance, in the context of a continuous glucose monitoring device, improper calibration can lead to inaccurate blood glucose level measurements. A person relying on such inaccurate blood glucose level measurements may consequently take too much insulin in reliance on the incorrect measurements, which can cause hypoglycemia-characterized by low blood sugar levels. Hypoglycemia can cause symptoms such as shakiness, dizziness, confusion, and even loss of consciousness. In severe situations, hypoglycemia is life-threatening.

Conversely, person relying on incorrect measurements who does not take enough insulin may suffer from hyperglycemia, which is characterized by high blood sugar levels. Hyperglycemia can cause symptoms such as fatigue and blurred vision. Over time, uncontrolled hyperglycemia can lead to serious complications such as nerve damage, kidney damage, and vision loss. Furthermore, if an analyte monitoring device is not calibrated properly, it may produce inconsistent results over time, making it difficult for individuals to effectively manage their analyte levels. Inconsistent readings also make it difficult for healthcare providers to accurately evaluate a person's health conditions and devise treatment plans accordingly.

Many analyte monitoring devices are designed to measure analyte levels using a probe (e.g., a sensor of a given configuration, such as a planar sensor, wire sensor, etc.) that subcutaneously contacts a wearing user's interstitial fluid. In some configurations, the probe includes an enzyme that creates an electrochemical reaction when exposed to an analyte (e.g., glucose, lactate, potential of hydrogen (pH), sodium, potassium, alcohol, etc.), which creates an electrical current that is indicative of an analyte concentration in the wearing user's interstitial fluid.

Conventional approaches to calibrating analyte monitoring devices involve separate fingerstick meters. For instance, a patient wanting to calibrate a glucose monitoring device using conventional calibration methods is forced to use separate supplies, such as a fingerstick glucose meter, glucose test strips, and a lancet device. The lancet device is used to prick a finger of the patient, which produces a small drop of blood. This small drop of blood is then applied to a glucose test strip, which in turn is inserted into the fingerstick glucose meter. The fingerstick glucose meter analyzes the blood and outputs a glucose reading on its display. The glucose reading displayed on the fingerstick glucose meter is then entered into the patient's glucose monitoring device, which adjusts its measurements based on the fingerstick calibration data. Thus, conventional approaches to calibrating analyte monitoring devices are physically invasive, uncomfortable, and require patients to carry dedicated calibration equipment that is separate from the analyte monitoring device being calibrated. Accordingly, it is desirable to calibrate analyte monitoring devices independent of (e.g., without) requiring patients to use separate calibration hardware.

To address these conventional shortcomings, some approaches to calibrating analyte monitoring devices involve placing an analyte sensor in a sensor bath having a known analyte concentration and monitoring an electrical current generated by the analyte sensor when placed in the bath. Advantageously, using known analyte concentration sensor baths enables analyte monitoring device calibration at a point of manufacture (e.g., factory calibration) and mitigates a need for patients to use separate calibration devices.

To measure the electrical current generated by an analyte monitoring device during a calibration procedure, some approaches may measure current across two points of contact with the analyte monitoring device. In such two-points-of-contact setups, one point of contact corresponds to an input voltage provided by a power source of the analyte monitoring device and another point of contact corresponds to an isolated ground component of the analyte monitoring device. Such two-points-of-contact setups are thus informed (e.g., via an external current monitor) of the electrical current and resulting analyte measurement generated by an analyte monitoring device, relative to a known analyte concentration.

These two-points-of-contact calibration systems and procedures, however, do not extend easily to calibration of analyte monitoring devices having small form factors. For instance, in small form factor electronic devices, making room for a ground electrical contact that is accessible by an external calibration system is a challenging task from a manufacturing perspective. One goal in manufacturing wearable analyte monitoring devices is to make the devices as small as possible while maintaining their functionality. Creating a ground electrical contact that is accessible by an external calibration system requires additional device space, particularly because smaller electrical contacts render it more difficult to create stable and reliable connections.

As another challenge facing two-points-of-contact calibration systems, ground electrical contacts accessible by an external calibration system often require additional layers in a printed circuit board design of the analyte monitoring devices, which add complexity to, and increase the cost of, manufacturing the analyte monitoring devices. As yet another drawback, adding an externally accessible ground contact can create electromagnetic interference issues if the analyte monitoring device is not designed properly, which further requires additional space to accommodate.

Thus, two-points-of-contact systems for calibrating analyte monitoring devices often require including components that prevent the analyte monitoring devices from achieving a smaller form factor. These device size constraints imposed by two-points-of-contact calibration systems, however, contrast with benefits of designing analyte monitoring devices to have smaller form factors. For instance, smaller form factors provide for more portable analyte monitoring devices that can be worn more comfortably (e.g., without interfering with a wearing user's movements or activities). Smaller analyte monitoring devices additionally provide the benefit of consuming less power than larger devices, which allows for increased battery life and reduces a frequency at which devices need to be recharged or replaced, the benefits of which are frustrated by two-points-of-contact calibration system requirements.

To determine calibration in a factory setting while permitting design of small form factor analyte monitoring devices, a calibration system is described that eliminates the need for analyte monitoring devices to include an electrical ground contact that is accessible by (e.g., directly connected to) the calibration system. As described herein, the calibration system is configured to simultaneously generate calibration results for multiple analyte monitoring devices. To do so, the calibration system includes a single ground electrode that is shared by the multiple analyte monitoring devices during the calibration evaluation process. Advantageously, the single ground electrode is not physically connected to the multiple analyte monitoring devices (e.g., via a local ground contact of each of the multiple analyte monitoring devices) during a calibration evaluation process. By including the single ground electrode that does not require contact or communicative coupling with an analyte monitoring device, the calibration system described herein enables for manufacture of analyte monitoring devices having smaller form factors and/or with fewer components.

The calibration system includes a voltage source that is configured to apply an input voltage to each of the multiple analyte monitoring devices via respective electrodes that are each connected to a sensor of a different one of the multiple analyte monitoring devices. During a calibration evaluation process, sensors of the multiple analyte monitoring devices and the single ground electrode are placed into a reference bath that contains a known concentration of an analyte to be measured by each of the multiple analyte monitoring devices.

The input voltage causes each sensor to generate an electrical current that is indicative of the concentration within the reference bath. The calibration system includes a current meter that monitors the electrical current generated by each of the multiple analyte monitoring devices. The current generated by a respective sensor of each analyte monitoring device is useable to identify a calibration parameter (e.g., a parameter that identifies a sensitivity of the corresponding sensor to the known analyte concentration of a given reference bath). Parameter value(s) derived for each of the multiple analyte monitoring devices during calibration via the reference baths are used to tune the corresponding analyte monitoring device such that the analyte monitoring device outputs reliable measurements of detected analyte concentration levels. In some implementations, one or more parameter values determined from calibrating at least one analyte monitoring device via the reference baths is used to calibrate one or more different analyte monitoring devices (e.g., other analyte monitoring devices of a common manufacturing batch, other analyte monitoring devices sharing one or more measurable parameters, and so forth).

The calibration system and techniques described herein thus enable manufacture and testing of analyte monitoring device having form factors of a small size. Consequently, the described systems and techniques enable design and manufacturing of analyte monitoring devices that are more portable, more comfortable (e.g., to a wearing user), more power efficient, and cheaper (e.g., with respect to manufacturing costs) without sacrificing an accuracy or reliability of results from factory calibration generated by the analyte monitoring device.

In some aspects, the techniques described herein relate to an apparatus configured to calibrate multiple analyte monitoring devices, the apparatus including a voltage source, a plurality of electrodes configured to receive an input voltage from the voltage source, each of the plurality of electrodes being connected to a different analyte monitoring device of the multiple analyte monitoring devices, and a single ground electrode configured to complete a circuit for each of the multiple analyte monitoring devices during calibration of the multiple analyte monitoring devices.

In some aspects, the techniques described herein relate to an apparatus, wherein the single ground electrode is not physically connected to the multiple analyte monitoring devices.

In some aspects, the techniques described herein relate to an apparatus, wherein each of the plurality of electrodes is connected to an analyte sensor of a respective one of the multiple analyte monitoring devices via a contact that conducts the input voltage from the voltage source to an analyte-sensing element of the analyte sensor.

In some aspects, the techniques described herein relate to an apparatus, wherein the analyte-sensing element is inserted subcutaneously into a user when the respective one of the multiple analyte monitoring devices is worn by the user.

In some aspects, the techniques described herein relate to an apparatus, wherein at least one of the plurality of electrodes is connected to an analyte sensor that is used to generate at least one calibration parameter for calibrating one or more different analyte sensors.

In some aspects, the techniques described herein relate to an apparatus, wherein the analyte-sensing element is from a manufacturing lot associated with a second analyte-sensing element, the second analyte sensing element configured to be inserted subcutaneously into a user.

In some aspects, the techniques described herein relate to an apparatus, further including a plurality of current meters, wherein each of the plurality of current meters monitors a current of the circuit for a corresponding one of the multiple analyte monitoring devices during calibration of the multiple analyte monitoring devices.

In some aspects, the techniques described herein relate to an apparatus, wherein the apparatus is configured such that inserting the single ground electrode into a reference bath causes an analyte-sensing element for each of the multiple analyte monitoring devices to be inserted into the reference bath during calibration of the multiple analyte monitoring devices.

In some aspects, the techniques described herein relate to an apparatus, wherein the reference bath includes a known concentration of an analyte to be measured by the multiple analyte monitoring devices.

In some aspects, the techniques described herein relate to an apparatus, wherein the input voltage from the voltage source causes each of the multiple analyte monitoring devices to generate an electrical current when an analyte-sensing element contacts a fluid that contains an analyte, wherein the electrical current indicates a concentration of the analyte within the fluid.

In some aspects, the techniques described herein relate to an apparatus, wherein the analyte-sensing element includes an enzyme that generates an electrochemical reaction when contacting the analyte, wherein the electrical current is generated from the electrochemical reaction.

In some aspects, the techniques described herein relate to a system configured to calibrate multiple analyte monitoring devices, the system including a reference bath containing a solution having a known analyte concentration, a calibration board including a plurality of electrodes, each of the plurality of electrodes being connected to a different analyte monitoring device of a plurality of analyte monitoring devices, and a single ground electrode that contacts the solution of the reference bath, a voltage source that applies voltage to the plurality of electrodes, and a plurality of current meters, each of the plurality of current meters monitoring a current flowing from a respective one of the plurality of electrodes to the single ground electrode via a respective one of the plurality of analyte monitoring devices.

In some aspects, the techniques described herein relate to a system, wherein the single ground electrode is not physically connected to the plurality of analyte monitoring devices.

In some aspects, the techniques described herein relate to a system, wherein the voltage source applies a constant voltage to the plurality of electrodes while the single ground electrode and an analyte-sensing element of each of the plurality of analyte monitoring devices contacts the solution of the reference bath.

In some aspects, the techniques described herein relate to a system, wherein the voltage source causes each of the plurality of analyte monitoring devices to generate the current flowing from the respective one of the plurality of electrodes to the single ground electrode, wherein the current indicates an observed analyte concentration of the solution of the reference bath.

In some aspects, the techniques described herein relate to a system, wherein each of the plurality of analyte monitoring devices includes an analyte-sensing element including an enzyme that generates an electrochemical reaction when contacting the known analyte concentration in the solution of the reference bath, wherein the current is generated from the electrochemical reaction.

In some aspects, the techniques described herein relate to a system, wherein the plurality of analyte monitoring devices are continuous glucose monitoring devices and the solution of the reference bath includes a known concentration of glucose.

In some aspects, the techniques described herein relate to a method including applying a voltage to an analyte monitoring device connected to a calibration board while a sensor of the analyte monitoring device and a single ground electrode of the calibration board are in contact with a reference bath, measuring, for the analyte monitoring device, a current that flows through the sensor to the single ground electrode as a result of applying the voltage, and generating calibration results for the analyte monitoring device based on the current.

In some aspects, the techniques described herein relate to a method, wherein the single ground electrode of the calibration board is not physically connected to the analyte monitoring device.

In some aspects, the techniques described herein relate to a method, wherein the current indicates an analyte concentration of a solution contained in the reference bath that is observed by the analyte monitoring device.

In some aspects, the techniques described herein relate to a method, wherein the sensor of the analyte monitoring device includes an enzyme that generates an electrochemical reaction when contacting an analyte included in a solution contained within the reference bath, wherein the current is generated by the electrochemical reaction.

In the following discussion, an exemplary environment is first described that is configured to employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

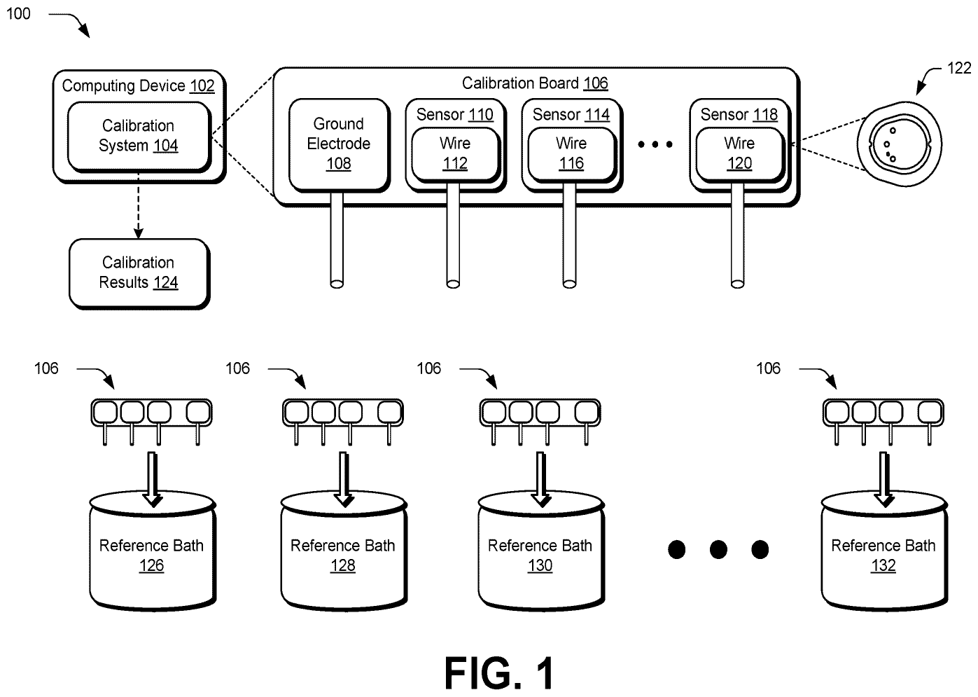

FIG. 1 is an illustration of an environment 100 in an example implementation that is operable for calibrating analyte monitoring devices in accordance with the systems and techniques described herein. The illustrated environment 100 includes a computing device 102, which is configured to calibrate multiple analyte monitoring devices.

The computing device 102 is configurable in a variety of manners in accordance with the described techniques. For instance, by way of example and not limitation, the computing device 102 is configured as a desktop computer, a laptop computer, a mobile device (e.g., a mobile phone or tablet device), a wearable device (e.g., a smart watch, one or more rings, bracelet or wristband, mouthguard, contact lenses, smart glasses, chest strap, ear buds, clothing, headphones, and the like), and so forth. In one or more implementations, the computing device 102 is representative of a dedicated device associated with an analyte device manufacturing entity, an analyte monitoring platform, a healthcare provider platform, or a combination thereof (e.g., a dedicated platform functional to obtain analyte measurements from an analyte monitoring device, perform various computations in relation to the analyte measurements, display information related to the analyte measurements, and so forth).

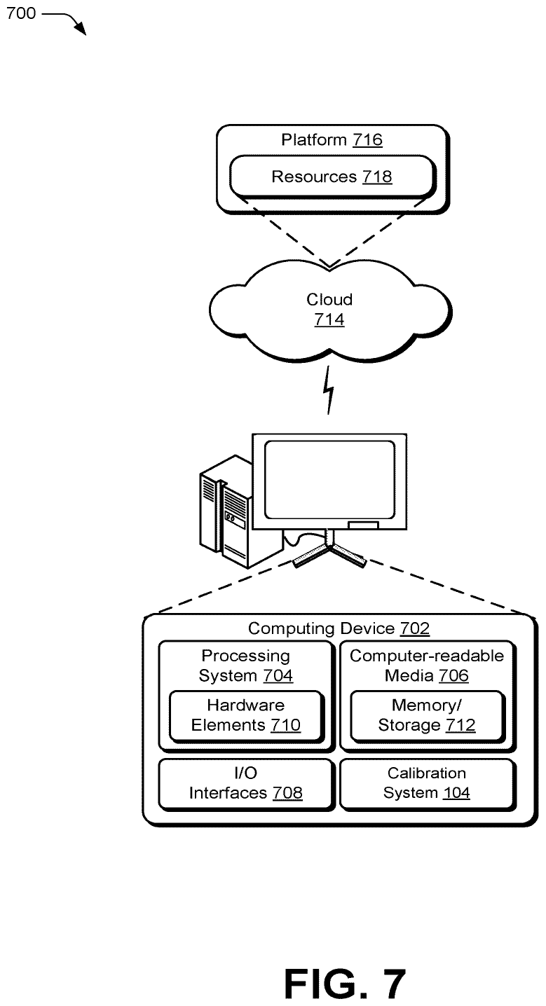

Alternatively or additionally, in some implementations the computing device 102 is representative of more than one device, as described in further detail below with respect to FIG. 7. In such scenarios, each of the multiple devices represented by computing device 102 may be capable of performing at least some of the same operations involved in calibrating analyte monitoring devices using the techniques described herein. Alternatively or additionally, different devices represented by the computing device 102 are configured with different capabilities (e.g., capabilities not supported by one or more other devices, which may be limited by hardware constraints and/or via computing instructions).

Functionality of the computing device 102 to calibrate analyte monitoring devices is represented in the illustrated example of FIG. 1 via the inclusion of calibration system 104. Although depicted as being included as part of the computing device 102, in accordance with one or more implementations the computing device 102 and the calibration system 104 represent separate devices that are communicatively coupled to one another. By way of example, the computing device 102 and the calibration system 104 communicate with one another using one or more of a wired connection, a wireless connection (e.g., via radio, cellular, Wi-Fi, Bluetooth, near-field communication (NFC), 5G, and the like), combinations thereof, and so forth.

To facilitate testing and calibration of analyte monitoring devices in accordance with the techniques described herein, the calibration system 104 is depicted as including a calibration board 106. The calibration board 106 includes a single ground electrode 108, which is useable to complete a circuit for monitoring electrical currents generated by individual analyte monitoring devices involved in a calibration evaluation process performed by the calibration system 104. For instance, in the illustrated example of FIG. 1, the calibration board 106 is depicted as being used to evaluate three different analyte monitoring devices during a calibration evaluation process.

Each of the three different analyte monitoring devices are represented by depicting a subset of their respective components as being coupled to the calibration board 106. For instance, a first analyte monitoring device is represented by sensor 110, which includes wire 112, a second analyte monitoring device is represented by sensor 114, which includes wire 116, and a third analyte monitoring device is represented by sensor 118, which includes wire 120. Although described herein and illustrated in the context of being configured as a “wire,” in some implementations individual sensors calibrated using the calibration board 106 include a component or are configured so to allow for dipping in one or more reference baths while being configured in a form factor other than a wire (e.g., planar sensors and so forth). For instance, in some implementations one or more of the sensor 110, sensor 114, or sensor 118 are manufactured in a form factor other than a wire that permits the inclusion of electrodes or analyte-responsive elements and/or areas that provide an electrical signal when exposed to a concentration of an analyte (e.g., via electrochemical means or otherwise). For the purposes of simplicity, however, the following description describes functionality of a sensor to react to an analyte concentration as being configured in a wire form factor.

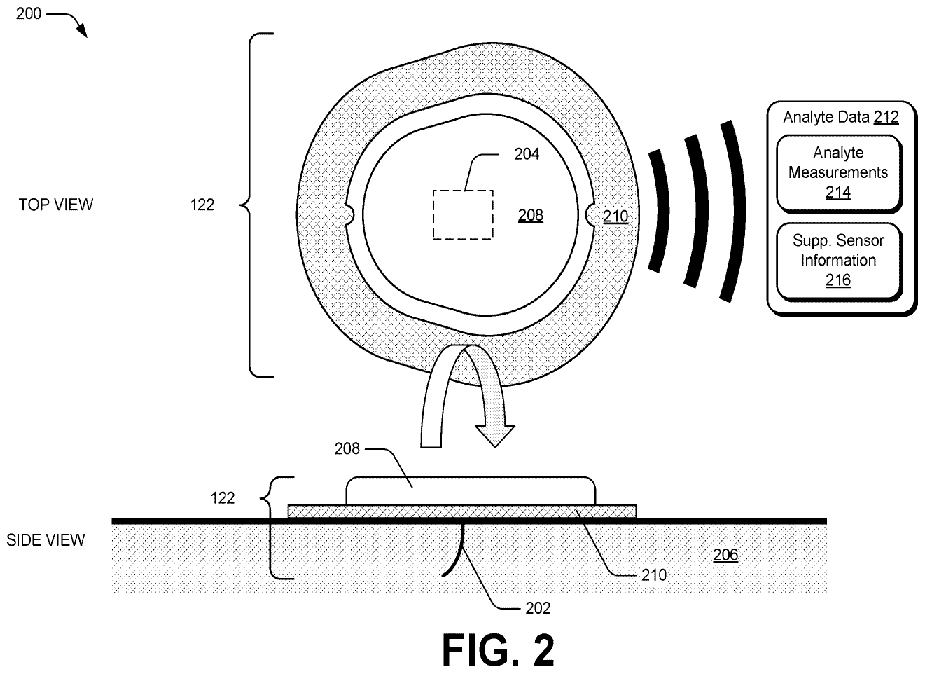

Each of the sensors connected to the calibration board 106 (e.g., sensor 110, sensor 114, and sensor 118) thus represents a component of a corresponding analyte monitoring device. For instance, in the illustrated example of FIG. 1, sensor 118 represents a component of analyte monitoring device 122, which is described in further detail below with respect to FIG. 2. Although depicted as being used to test three different analyte monitoring device sensors during a calibration evaluation process, the calibration board 106 is configurable to support any number (e.g., one or more) analyte monitoring device sensors, as indicated by the ellipses separating sensor 114 and sensor 118.

In one or more implementations, the analyte monitoring device 122 is a wearable, such that it is configured to be worn by a person while the analyte monitoring device 122 performs various operations. Additionally or alternatively, the analyte monitoring device 122 performs one or more operations before or after being worn by a person. The analyte monitoring device 122 is thus configured to provide measurements of an analyte of a person (e.g., measurements of a person's glucose levels). For instance, the analyte monitoring device 122 may be configured with one or more analyte sensors (e.g., one or more glucose sensors) that detect one or more signals indicative of the analyte in a person and enable generation of analyte measurements or estimations (e.g., estimated analyte values). Those analyte measurements (e.g., glucose measurements) may correspond to or otherwise be packaged for communication to one or more computing devices or a medicament delivery system as analyte data, examples of which are described in further detail below with respect to FIG. 2.

Although a wearable analyte monitoring device is referenced herein, the described analyte monitoring device calibration system and techniques are useable with other configurations, such as non-wearable analyte devices (e.g., blood glucose meters requiring finger sticks), implants, patches, and so forth. Additionally or alternatively, the described analyte monitoring device calibration system and techniques are useable with sensors that produce data about different physiological phenomena from an analyte (e.g., multiple analytes, temperature, electrical signals, photonic events, heart rate, heart rate variability, blood pressure, and so forth).

In at least one implementation, the analyte monitoring device 122 is part of a glucose monitoring system. As an example, the analyte monitoring device 122 may be configured as a continuous glucose monitoring (“CGM”) system (e.g., a wearable CGM system). As used herein, the term “continuous” when used in connection with analyte monitoring may refer to an ability of a device to produce analyte measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device, and so forth. In some implementations, the analyte monitoring device 122 provide analyte measurements when requested.

For example, the analyte monitoring device 122 may produce a current analyte measurement responsive to a request from a computing device that is communicatively coupled to the analyte monitoring device 122 (e.g., computing device 102). This request may be initiated in a variety of different ways, such as responsive to user input to a user interface (e.g., of an application) displayed by the computing device 102 (and/or a different device), responsive to placement of the computing device 102 within a threshold proximity of the analyte monitoring device 122, responsive to the computing device 102 making physical contact with the analyte monitoring device 122, responsive to a request from an application implemented at the computing device 102, combinations thereof, and so forth. This functionality, along with further aspects of the configuration of the analyte monitoring device 122, is discussed in more detail in relation to FIG. 2. In addition to producing analyte measurements, the analyte monitoring device 122 represents functionality to transmit produced analyte measurements (e.g., to the computing device 102 and/or to an analyte monitoring platform or health monitoring platform).

The calibration system 104 generates calibration results 124 for each of the analyte monitoring devices connected to the calibration board 106 during a calibration evaluation process. To do so, the calibration system 104 is configured to place the single ground electrode 108 and a sensor wire, or other analyte-sensing element, of each analyte monitoring device 122 (e.g., wire 112, wire 116, and wire 120) in one or more reference baths. Specifically, in the illustrated example of FIG. 1, a calibration evaluation process involves generating calibration results 124 that describe each analyte monitoring device sensor's reaction to placement in reference bath 126, reference bath 128, reference bath 130, and reference bath 132. Although depicted in the illustrated example as involving four different reference baths, a calibration evaluation process as described herein is configurable to include any number of reference baths (e.g., one or more reference baths), as indicated by the ellipses between reference bath 130 and reference bath 132.

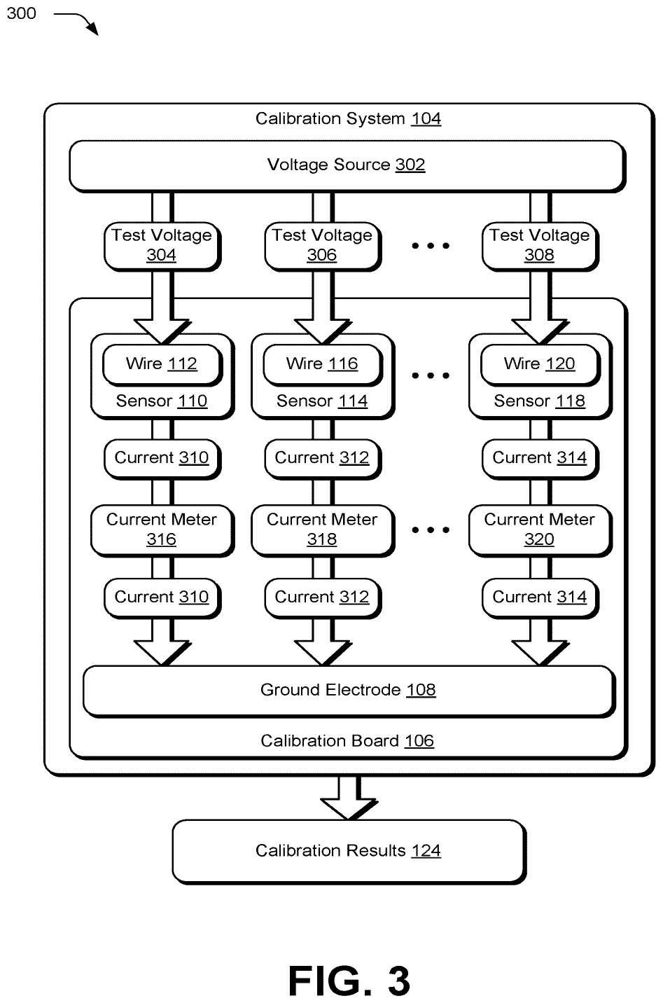

As described in further detail below with respect to FIGS. 3-5, in some implementations each analyte-sensing element of an analyte monitoring device 122 (e.g., sensor wire) includes an enzyme that causes an enzymatic electrochemical reaction when a voltage is applied to the analyte-sensing element and the when the analyte-sensing element is exposed to a certain analyte (e.g., one or more analytes that the analyte monitoring device 122 is designed to detect). For instance, in an example implementation where the analyte monitoring device 122 is representative of a continuous glucose monitoring device, the wire 112, the wire 116, and the wire 120 each include an enzyme (e.g., glucose oxidase) that, when exposed to glucose, catalyzes the oxidization of glucose and produces a chemical compound (e.g., hydrogen peroxide). The production of this chemical compound as a result of oxidization generates an electrical current on the wire (e.g., the wire 112, the wire 116, and the wire 120), which provides a signal indicative of a concentration of glucose in the fluid (e.g., reference bath fluid, interstitial fluid, etc.) to which the wire is exposed. This signal is converted by the analyte monitoring device 122 into an analyte measurement (e.g., a glucose value expressed in mg/dL) which is output by the analyte monitoring device 122 to inform a user of the analyte monitoring device 122 as to their analyte measurement.

In one example implementation, a signal provided by the electrical current generated by a sensor's analyte-sensing element (e.g., when a sensor wire is under voltage and exposed to an analyte) is linear, such that a change in analyte concentration is linearly proportional to a change in the signal (e.g., an electrical current expressed in picoamps). In such an example implementation, different analyte concentrations included in the reference bath 126, reference bath 128, reference bath 130, and reference bath 132 are equally spaced, such that the calibration system 104 evaluates whether electrical currents generated by a given analyte-sensing element (e.g., wire 112) are linearly proportional when interpolated across the analyte-sensing element's exposure to different reference baths. In another example implementation, a signal provided by the electrical current generated by an analyte-sensing element (e.g., when a sensor wire is under voltage and exposed to an analyte) is non-linear.

For instance, consider a specific example calibration evaluation process where the reference baths of FIG. 1 are configured to evaluate performance of analyte monitoring devices configured for continuous glucose monitoring. In this specific example calibration evaluation process, reference bath 126 includes a solution having a glucose concentration that corresponds to a lower bound of a range of glucose concentrations detectable by the glucose sensor(s) being calibrated (e.g., a minimum glucose value that is reportable by a glucose monitoring device with an acceptable degree of confidence). With respect to this specific example calibration process, reference bath 132 includes a solution having a glucose concentration that corresponds to an upper bound of a range of glucose concentrations detectable by the glucose sensor(s) being calibrated (e.g., a maximum glucose value that is reportable by the glucose monitoring device with an acceptable degree of confidence). Continuing this example calibration process, intermediate reference baths (e.g., reference bath 128 and reference bath 130) each include solutions having respective glucose concentrations that are distributed between the glucose concentration of reference bath 126 and the glucose concentration of reference bath 132. For instance, reference bath 128 includes a glucose concentration that is proportionally (e.g., equally) different relative to glucose concentrations of adjacent reference bath. As an example, a difference between the glucose concentrations of reference bath 126 and reference bath 128 is the same as a difference between glucose concentrations of reference bath 128 and reference bath 130, which in turn is the same as a difference between glucose concentrations of reference bath 130 and reference bath 132.

Given the common differences in glucose concentrations between adjacent reference baths, the example calibration evaluation process is designed to produce a linear change in currents generated by each sensor wire (e.g., wire 112, wire 116, and wire 120) when progressively moved to different reference baths. That is, a change in signal that represents the electrical current generated by a sensor wire is expected to be linear as the sensor wire is first exposed to reference bath 126, then to reference bath 128, then to reference bath 130, and finally to reference bath 132.

Given the knowledge of analyte concentrations (e.g., glucose concentrations) in each reference bath and the signal (e.g., picoamp current) for each analyte monitoring device 122 analyte-sensing element (e.g., sensor wire) as measured by the calibration board 106, the calibration system 104 is configured to expect a linear response from the analyte monitoring device 122 when operating properly (e.g., when free from manufacturing defect). The calibration system 104 thus outputs calibration results 124 that indicate whether each analyte monitoring device 122 analyte-sensing element responds as expected during the calibration evaluation process. For instance, in context of the specific example calibration evaluation process described above, the calibration results 124 indicate that the analyte monitoring devices represented by sensor 110 and sensor 118 are operating properly (e.g., operating as intended or expected) in response to detecting linear changes in currents measured in the wire 112 and the wire 120 when exposed to the reference bath 126, reference bath 128, reference bath 130, and reference bath 132.

Continuing this example, the calibration results 124 indicate that the analyte monitoring device represented by the sensor 114 is not operating properly in response to detecting non-linear or otherwise inconsistent changes in currents measured in the wire 116 when exposed to the reference bath 126, reference bath 128, reference bath 130, and reference bath 132. The calibration results 124 are thus representative of information describing any suitable metric involved in a calibration evaluation process, such as respective reference bath analyte concentrations, time spent in each reference bath, current(s) observed in a analyte-sensing element when exposed to each reference bath solution, environmental conditions associated with the process (e.g., reference bath temperature, surrounding air temperature or humidity, oxygen levels, PH levels, combinations thereof, and so forth). In this manner, the calibration results 124 are representative of binary (e.g., success or failure) results for each analyte-sensing element involved in a calibration process as well as detailed metrics describing conditions of the calibration process and observed results. The calibration system 104 is configured to output the calibration results 124 to the computing device 102 (e.g., for storage and/or display at the computing device 102), to one or more other computing devices, or a combination thereof. A further description of the calibration system 104 evaluating analyte monitoring devices is provided below with respect to FIGS. 3-5.

In the context of a device configured for continuously measuring an analyte (e.g., glucose) and generating analyte data describing such measurements, consider the following description of FIG. 2.

FIG. 2 depicts an example 200 of an implementation of the analyte monitoring device 122 of FIG. 1 in greater detail. In particular, the illustrated example 200 includes a top view and a corresponding side view of the analyte monitoring device 122. It is to be appreciated that the analyte monitoring device 122 may vary in implementation from the following description in various aspects without departing from the spirit or scope of the described techniques.

In the example 200, the analyte monitoring device 122 is illustrated to include an analyte sensor 202 (e.g., a glucose sensor) and a sensor module 204. The analyte sensor 202 is depicted in the side view having been inserted subcutaneously into skin 206 (e.g., skin of a person wearing the analyte monitoring device 122). The sensor module 204 is approximated in the top view as a dashed rectangle. The analyte monitoring device 122 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data (e.g., over a wireless connection to a medicament delivery system, a medical provider system such as a caregiver or hospital system, a computing device of a user wearing the analyte monitoring device 122, a data analytics platform, combinations thereof, and so forth) may also be housed or otherwise implemented within the housing of the transmitter 208. In the illustrated example of FIG. 2, the analyte monitoring device 122 further includes adhesive pad 210 that adheres the analyte monitoring device 122 to the skin 206.

In operation, the analyte sensor 202 and the adhesive pad 210 are assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, in some implementations the transmitter 208 is incorporated as part of the application assembly, such that the analyte sensor 202, the adhesive pad 210, and the transmitter 208 (with the sensor module 204) are all applied at once to the skin 206. In one or more implementations, this application assembly is applied to the skin 206 using a separate sensor applicator (not shown). Unlike the finger sticks required by conventional blood glucose meters, user-initiated application of the analyte monitoring device 122 with a sensor applicator is nearly painless and does not require the withdrawal of blood. Moreover, the sensor applicator generally enables a person wearing the analyte monitoring device 122 to embed the analyte sensor 202 subcutaneously into the skin 206 without the assistance of a clinician or healthcare provider.

In some implementations the analyte monitoring device 122 is removable by peeling the adhesive pad 210 from the skin 206. The analyte monitoring device 122 and its various components as illustrated are simply one example form factor, and the analyte monitoring device 122 and its components is configurable using different form factors without departing from the spirit or scope of the described techniques.

In operation, the analyte sensor 202 is communicably coupled to the sensor module 204 via at least one communication channel which can be a wireless connection or a wired connection. Communications from the analyte sensor 202 to the sensor module 204 or from the sensor module 204 to the analyte sensor 202 can be implemented actively or passively and these communications can be analog or digital.

The analyte sensor 202 is representative of a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 includes glucose oxidase which reacts with glucose and oxygen to form hydrogen peroxide that is electrochemically detectable by the sensor module 204 which may include an electrode. In one example, the analyte sensor 202 is configured as or includes a glucose sensor configured to detect analytes in blood, interstitial fluid, or other bodily fluids that are indicative of glucose level using one or more measurement techniques. In one or more implementations, the analyte sensor 202 is configured to detect analytes in the blood, interstitial fluid, or other bodily fluids that are indicative of other markers, such as lactate levels, ketones, or ionic potassium, which may improve accuracy in identifying or predicting glucose-based events (e.g., hyperglycemia or hypoglycemia). Additionally or alternatively, the analyte monitoring device 122 includes additional sensors and/or architectures to the analyte sensor 202 configured to detect those analytes indicative of the other markers.

In some examples, the sensor module 204 and the analyte sensor 202 are configured to detect a single analyte (e.g., glucose). In other examples, the sensor module 204 and the analyte sensor 202 are configured to use diverse sensing modes to detect multiple analytes (e.g., ionic sodium, ionic potassium, carbon dioxide, glucose, insulin, and so forth). Alternatively or additionally, the analyte monitoring device 122 includes multiple sensors to detect not only one or more analytes (e.g., ionic sodium, ionic potassium, carbon dioxide, glucose, insulin, and so forth) but also one or more environmental conditions (e.g., temperature, moisture, movement, and so forth). Thus, the sensor module 204 and the analyte sensor 202 (as well as any additional sensors) are configured to detect the presence of one or more analytes, the absence of one or more analytes, and/or changes in one or more environmental conditions. As noted above, the analyte monitoring device 122 is configured to produce data describing a single analyte (e.g., glucose) or multiple analytes in accordance with one or more implementations.

In one or more implementations, the sensor module 204 includes a processor and memory (not depicted). The sensor module 204, by leveraging the processor, generates analyte measurements 214 based on the communications with the analyte sensor 202 that are indicative of the above-discussed changes. Based on the above-noted communications from the analyte sensor 202, the sensor module 204 is further configured to generate communicable packages of data that include at least one analyte measurement 214. During calibration of the analyte sensor 202, the communicable packages of data that include at least one analyte measurement 214 are output to the calibration system 104 for use in generating calibration results 124 for the analyte monitoring device 122. In the example 200, the analyte measurements 214 represent these packages of data. Additionally or alternatively, the sensor module 204 configures the analyte data 212 to include additional data, including, by way of example, supplemental sensor information 216. The supplemental sensor information 216 is representative of information including a sensor identifier, a sensor status, temperatures that correspond to the analyte measurements 214, measurements of other analytes that correspond to the analyte measurements 214, and so forth. It is to be appreciated that supplemental sensor information 216 may include a variety of data that supplements at least one analyte measurement 214 without departing from the spirit or scope of the described techniques.

In some implementations, supplemental sensor information 216 includes orientation data that describes forces measured by an accelerometer of the analyte monitoring device 122 (not pictured). In such an example implementation, the supplemental sensor information 216 is useable by a data analytics platform to determine a location of the analyte sensor 202's insertion site on a person wearing the analyte monitoring device 122. In this example implementation, the supplemental sensor information 216 includes forces measured by an accelerometer that are useable by a data analytics platform to identify one or more characteristic force patterns. Each characteristic force pattern is associated with an insertion site location for the analyte sensor 202, which is useable to derive an insertion site of the analyte monitoring device 122 being worn by a person based on a characteristic force pattern represented in the supplemental sensor information 216.

In some implementations, supplemental sensor information 216 includes light data describing reflected light measured by a photodiode sensor of the analyte monitoring device 122 (not pictured). For example, the analyte monitoring device 122 includes light emitting diodes (LEDs) that are configured to transmit light directed at skin 206 disposed around the analyte sensor 202's insertion site. The light transmitted by the LEDs reflects from the skin 206 disposed around the analyte sensor 202's insertion site, and this reflected light is received by the photodiode sensor. The analyte monitoring device 122 is thus configured to generate supplemental sensor information 216 that includes light data describing light reflected from the skin 206 disposed around the analyte sensor 202's insertion site.

Light data is useable (e.g., by a data analytics platform) to identify characteristic light patterns that are indicative of an insertion site location or an insertion site anomaly for the analyte sensor 202. For example, the anomaly of the insertion site is a tattoo, a scar tissue, a skin irritation, etc. A data analytics platform is thus able to process the supplemental sensor information 216 to identify an insertion site location or insertion site anomaly as corresponding to most similar characteristic light pattern to a light pattern described by the light data.

In some implementations, supplemental sensor information 216 describing an insertion site location or insertion site anomaly of the analyte sensor 202 influences a manner in which sensor data (e.g., the analyte measurements 214) are interpreted by a data analytics platform. For instance, in some implementations a particular insertion site represented by the supplemental sensor information 216 corresponds to an insertion site that is not intended or recommended by a manufacturer of the analyte monitoring device 122. In such implementations, a data analytics platform is enabled to identify that information represented in the analyte data 212 should be amplified (e.g., increased or decreased) to offset inaccuracies in the analyte data 212 that result from inserting the analyte sensor 202 in the suboptimal insertion site location. Consequently, a data analytics platform is enabled to generate modified sensor data from received analyte data 212 that represents, for example, analyte measurements 214 that likely would have been measured by the analyte sensor 202 if the analyte sensor 202 was inserted at a recommended insertion site location (e.g., located at a person's abdomen instead of the suboptimal insertion site location).

In some implementations, the supplemental sensor information 216 represents other patient characteristics, such as patient heart rate data. For instance, patient heart rate data refers to the heart rate, heart rate variability, oxygen saturation, and so forth, of a person wearing the analyte monitoring device 122. As used herein, the term “heart rate variability” refers to variations in time intervals between heartbeats and these variations can indicate corresponding variations in the analyte levels of a person wearing the analyte monitoring device 122. Such example heart rate data is ascertained by a heart rate sensor, pulse oximeter, or other sensor (not pictured) integrated into the analyte monitoring device 122.

In implementations where the analyte monitoring device 122 is configured for wireless transmission, the transmitter 208 transmits the analyte data 212 as a stream of data to a computing device. Alternatively or additionally, the sensor module 204 buffers the analyte measurements 214 and/or the supplemental sensor information 216 (e.g., in memory of the sensor module 204 and/or other physical computer-readable storage media of the analyte monitoring device 122) and causes the transmitter 208 to transmit the buffered sensor data later at various regular or irregular intervals, e.g., time intervals (approximately every second, approximately every thirty seconds, approximately every minute, approximately every five minutes, approximately every hour, and so on), storage intervals (when the buffered analyte measurements 214 and/or supplemental sensor information 216 reach a threshold amount of data or a number of measurements), and so forth. In implementations the analyte monitoring device 122 can vary in numerous ways from the example described above without departing from the spirit or scope of the described techniques.

Having considered an example of an environment and an example of a wearable analyte monitoring device, consider now a discussion of some examples of details of the systems and techniques for calibrating wearable analyte monitoring devices in accordance with one or more implementations.

### Analyte Monitoring Device Calibration

FIG. 3 depicts an example 300 of a system to calibrate analyte monitoring devices in accordance with the techniques described herein. To do so, the illustrated example 300 is depicted as including the calibration system 104.

In this example 300, the calibration system 104 includes a voltage source 302. The voltage source 302 is a component or device of the calibration system 104 that is configured to provide a voltage output to one or more analyte monitoring devices being calibrated by the calibration system 104, such as the analyte monitoring devices represented by sensor 110, sensor 114, and sensor 118. In implementations, the voltage source 302 is configured to apply a fixed voltage, a variable voltage, or combinations thereof, to analyte monitoring devices during a calibration evaluation process.

The voltage source 302 is thus representative of a power supply from one or more power sources (e.g., wall outlet), a battery, a generator, a solar cell, a fuel cell, a voltage regulator, or combinations thereof. Although depicted in the illustrated example 300 as being included as part of the calibration system 104, in some implementations the voltage source 302 represents a component of the calibration system 104 to receive power from an external source (e.g., a source other than the calibration system 104) and convert the power to a defined voltage for use in calibrating analyte monitoring devices.

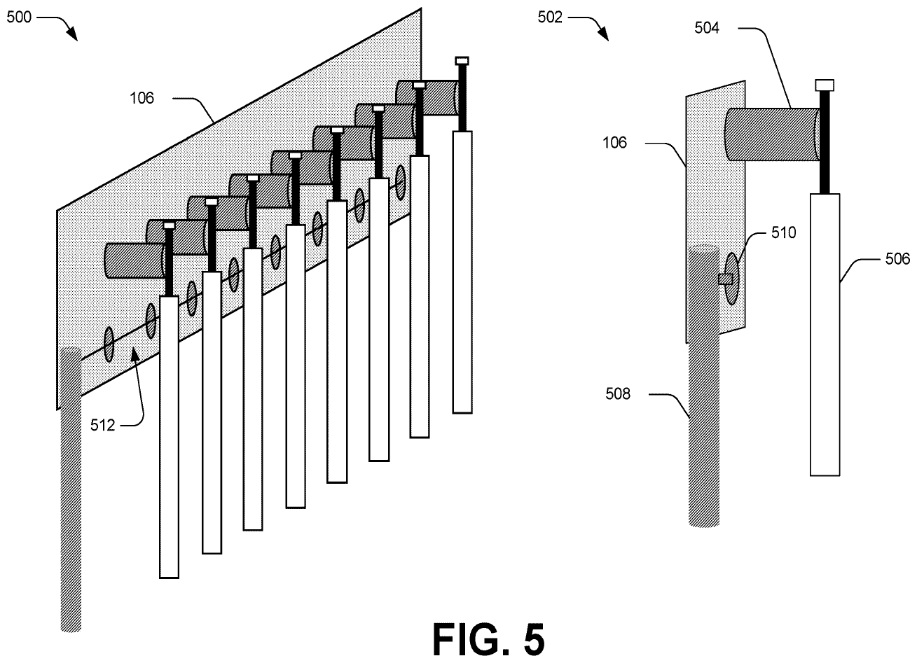

In the illustrated example 300, the voltage source 302 is depicted as providing test voltage 304 to wire 112 of sensor 110, providing test voltage 306 to wire 116 of sensor 114, and providing test voltage 308 to wire 120 of sensor 118. In implementations, the test voltage 304, the test voltage 306, and the test voltage 308 each represent a common (e.g., the same) voltage, such that the wire 112, the wire 116, and the wire 120 are caused to generate current from a same voltage when submerged (e.g., inserted into) a reference bath (e.g., the reference bath 126, the reference bath 128, the reference bath 130, and/or the reference bath 132). In some implementations, the test voltage 304, the test voltage 306, and the test voltage 308 each represent a constant voltage applied by the voltage source 302, such that each analyte monitoring device 122 being tested via the calibration board 106 becomes a common ground potentiostat via connection of its respective wire (e.g., the wire 112, the wire 116, or the wire 120) to both the voltage source 302 and the ground electrode 108, as described in further detail below with respect to FIG. 5. In alternative examples, one or more different voltages may be applied to the different sensors (e.g., a first voltage is applied to sensor 110, a second voltage is applied to sensor 114, and a third voltage is applied to sensor 118).

A potentiostat is an electronic device used in electrochemistry to control the voltage of an electrode and measure the resulting current. Potentiostats generally consist of a three-electrode system, where the electrodes are commonly referred to as a working electrode, a reference electrode, and a counter electrode. The working electrode of a potentiostat is an electrode of interest, from which resulting current is measured (e.g., the electrode where the electrochemical reaction is taking place), while the reference electrode maintains a constant potential and the counter electrode provides a means for completing the electrical circuit. With respect to the example 300, which includes three potentiostats, the reference electrode for each potentiostat is represented by the voltage source 302 and the counter electrode is represented by the ground electrode 108. The wire 112 represents the working electrode for a first potentiostat, the wire 116 represents the working electrode for a second potentiostat, and the wire 120 represents the working electrode for a third potentiostat.

The calibration system 104 thus applies a voltage to each potentiostat (e.g., the test voltage 304, the test voltage 306, and the test voltage 308) and measures a resulting current while maintaining a constant potential difference between the voltage source 302 and the ground electrode 108. For instance, maintaining a constant potential difference via the application of test voltage 304 to wire 112 while the wire 112 is inserted into a reference bath (e.g., in contact with a fluid having a defined analyte concentration) between the voltage source 302 and the ground electrode 108 causes the wire 112 to generate current 310. Similarly, test voltage 306 causes wire 116 to generate current 312 and test voltage 308 causes wire 120 to generate current 314.

In order to investigate the behavior of analyte monitoring devices' analyte-sensing elements (e.g., of sensor 110, sensor 114, and sensor 118) as the analyte-sensing elements are transferred between reference bath 126, reference bath 128, reference bath 130, and reference bath 132, the calibration system 104 includes at least one current meter that is configured to measure the current 310, the current 312, and the current 314. For instance, in the illustrated example 300, the calibration system 104 is depicted as including current meter 316 to monitor the current 310, current meter 318 to monitor the current 312, and current meter 320 to monitor the current 314.

In implementations, each current meter represents a component of the calibration system 104 that is placed in series with a circuit for which current is being monitored (e.g., current meter 316 is placed in series with the circuit of the potentiostat with electrodes represented by voltage source 302, wire 112, and ground electrode 108). The current meter 316, the current meter 318, and the current meter 320 are thus representative of any one or combination of devices suitable to monitor electrical current. For instance, in some implementations a current meter employed by the calibration system 104 is an ammeter, which is a device that is designed to measure electrical current in (e.g., in amperes, picoamps, etc.) and has a low resistance to minimize an impact of the meter on the circuit.

Alternatively or additionally, a current meter employed by the calibration system 104 is a current probe, which monitors current of a circuit by detecting a magnetic field generated by the current flowing through a conductor. Alternatively or additionally, a current meter employed by the calibration system 104 is a shunt resistor, which is a low-resistance resistor placed in parallel with a circuit to monitor current in the circuit (e.g., using Ohm's law to calculate current from measured voltage drop across the shunt resistor). Alternatively or additionally, a current meter employed by the calibration system 104 is a hall effect sensor, which operates by detecting a magnetic field generated by a current flowing through a conductor, either connected in series or parallel with a circuit being monitored. Alternatively or additionally, a current meter employed by the calibration system 104 is an oscilloscope, a multimeter, or combinations thereof.

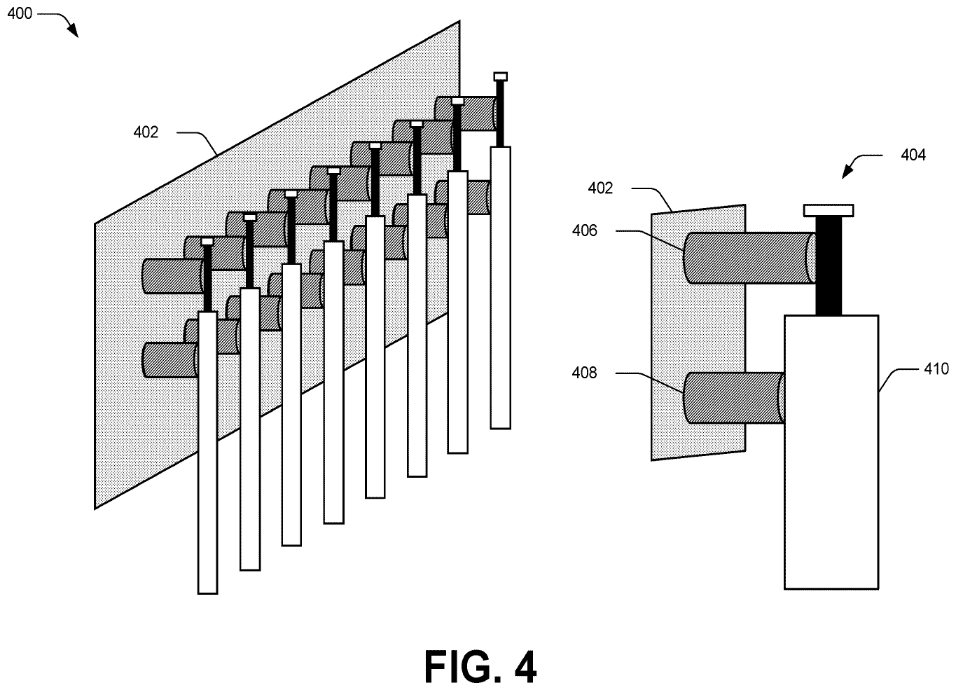

The calibration system 104 is configured to output information measured by the current meter 316, the current meter 318, and the current meter 320 (e.g., information expressing value(s) for each of the current 310, current 312, and current 314 as part of the calibration results 124. Having considered examples of a calibration system 104 useable to generate calibration results 124 for multiple analyte-sensing elements using the techniques described herein, consider an example configuration of the calibration system 104 in further detail with respect to the illustrated example of FIG. 5. To further clarify differences between the calibration system 104 and a two-points-of-contact calibration system, FIG. 4 first depicts an example of a two-points-of-contact calibration apparatus.

FIG. 4 depicts an example 400 of a two-points-of-contact apparatus involved in calibrating analyte monitoring devices, where a calibration board is connected to an analyte-sensing element being calibrated via two points of contact. In the illustrated example 400, calibration board 402 is depicted as supporting analyte-sensing elements for multiple analyte monitoring devices to be tested during a calibration evaluation process. In some implementations, the calibration board 402 is used as part of a calibration evaluation process that involves testing the analyte monitoring devices using reference baths as generally described above with respect to FIG. 1. View 404 provides a detailed view of the calibration board 402, which depicts how a single analyte monitoring device's analyte-sensing element is connected to the calibration board 402 during the calibration evaluation process.

As depicted at view 404, the calibration board 402 includes two points of contact that directly connect the analyte-sensing element to the calibration board 402, depicted as a voltage contact 406 and a ground contact 408. The voltage contact 406 and the ground contact 408 are each physically in contact with a portion of an analyte-sensing element, such as a wire 410 of an analyte monitoring device. In implementations, at least a portion of the wire 410 includes an enzyme configured to oxidize when the enzyme contacts an analyte for which the analyte monitoring device that includes the wire 410 is designed to measure. The wire 410 is configured to receive an input voltage from the voltage contact 406 during the calibration evaluation process and the ground contact 408 is depicted as physically contacting the wire 410. Thus, using two points of contact (e.g., the voltage contact 406 and the ground contact 408), the calibration board 402 is configured to create a complete circuit via which current can be monitored while the analyte monitoring device having wire 410 is tested during a calibration evaluation process. As evidenced by the illustrated example of FIG. 4, however, the ground contact 408 requires that the analyte monitoring device be manufactured in a manner that provides an available point of contact with the ground contact 408. Although not depicted in the illustrated example of FIG. 4, the point of contact between the ground contact 408 and the wire 410 is often located within an external housing of the analyte monitoring device.

As such, two-points-of-contact calibration system architectures necessitate that analyte monitoring devices be constructed as having a form factor of a size that enables for an electrical contact (e.g., pogo pin contact) between the ground contact 408 and an internal component of the analyte monitoring device (e.g., a portion of the wire 410 enclosed within a housing of the analyte monitoring device). Consequently, two-points-of-contact calibration system architectures may prevent an analyte monitoring device from achieving a smallest possible form factor and may require design and manufacture of analyte monitoring devices that are more cumbersome, expensive to manufacture, and less energy efficient relative to analyte monitoring devices that can be calibrated using the systems and techniques described herein. Having considered a two-points-of-contact calibration system, consider an example configuration of the calibration system 104 in further detail with respect to the illustrated example of FIG. 5.

FIG. 5 depicts an example 500 of an apparatus used by the calibration system 104 to calibrate analyte monitoring devices in accordance with the techniques described herein. The example 500 is depicted as including calibration board 106, which is illustrated as supporting analyte-sensing elements for multiple analyte monitoring devices (e.g., multiple instances of the analyte monitoring device 122) for testing during a calibration evaluation process, such as the calibration evaluation process that involves testing the analyte monitoring devices using reference baths as generally described above with respect to FIG. 1. View 502 provides a detailed view of the calibration board 106, which depicts how a single analyte monitoring device 122 is connected to the calibration board 106 via a single point of contact during the calibration evaluation process.

As depicted at view 502, the calibration board 106 includes an electrode 504, which represents a component of the calibration board 106 that provides (e.g., conducts) an input voltage (e.g., test voltage 304) from voltage source 302 to an analyte-sensing element (e.g., wire 506) of an analyte monitoring device (e.g., analyte monitoring device 122) being tested during the calibration evaluation process. In implementations, the electrode 504 is representative of any type of electrical contact of a material that conducts electrical voltage, such as a copper pogo pin connection, and is configured to receive a positive input voltage or a negative input voltage from the voltage source 302. The calibration board 106 additionally includes a single ground electrode 508, which represents an example configuration of the ground electrode 108. Notably, in contrast to the illustrated example 400, the ground electrode 508 is connected to the calibration board 106 via contact 510 and is not physically connected to the wire 506. The ground electrode 508 is oriented such that when the wire 506 is inserted into a reference bath (e.g., reference bath 126, reference bath 128, reference bath 130, and/or reference bath 132), the ground electrode 508 is also inserted into and/or otherwise makes contact (e.g., forming and/or in conductive contact with a container that holds the reference bath fluid) with the reference bath, thus creating a complete circuit via which the calibration board 106 can monitor current flowing through the wire 506. The illustrated example 500 demonstrates how the ground electrode 508 is connected to ground contacts for each analyte monitoring device analyte-sensing element connected to the calibration board 106, represented via the connection 512 in the illustrated example of FIG. 5. In this manner, calibration system 104 enables for testing and calibrating analyte-sensing elements without requiring that analyte monitoring devices which include the analyte-sensing elements be manufactured or otherwise designed to provide a ground contact between the analyte-sensing element and the calibration board 106. Thus, the calibration system 104 allows for design and manufacture of analyte monitoring devices having extremely small form factors, which are consequently less cumbersome, easier to manufacture, require less materials, and/or more energy efficient.

Having discussed exemplary details of systems and techniques for calibrating analyte monitoring devices, consider now some examples of procedures to illustrate additional aspects of the techniques.

### Example Procedures

This section describes examples of procedures for calibrating analyte monitoring devices. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a calibration system, such as the calibration system 104 that makes use of at least one of the calibration board 106, the ground electrode 108, one or more reference baths (e.g., reference bath 126, reference bath 128, reference bath 130, and reference bath 132), the voltage source 302, or one or more current meters (e.g., current meter 316, current meter 318, and current meter 320).

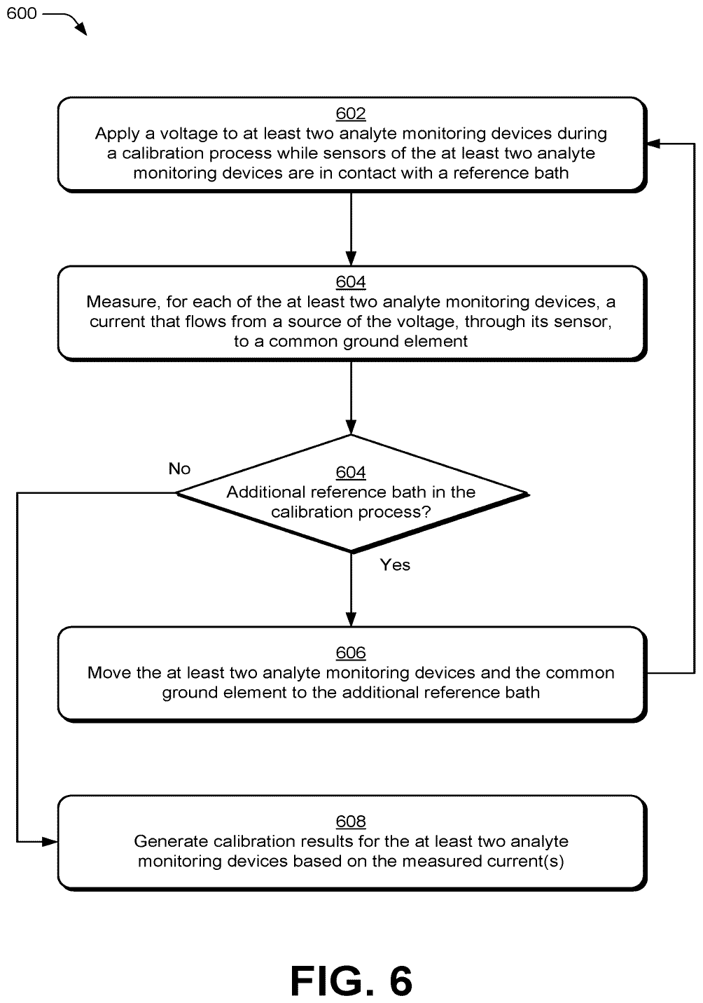

FIG. 6 depicts a procedure 600 in an example implementation of calibrating analyte monitoring devices using the calibration system 104. To begin, a voltage is applied to at least two analyte monitoring devices during a calibration process while sensors of the at least two analyte monitoring devices are in contact with a reference bath (block 602). The calibration system 104, for instance, uses voltage source 302 to apply test voltage 304 to wire 112, apply test voltage 306 to wire 116, and test voltage 308 to wire 120 while the wire 112, the wire 116, the wire 120, and the ground electrode 108 are in contact with a solution having a known analyte concentration (e.g., in contact with a fluid included in reference bath 126).

For each of the at least two analyte monitoring devices, a current that flows from a source of the voltage, through a sensor of the analyte monitoring device, to a common ground element is measured (block 604). The calibration system 104, for instance, leverages current meter 316 to monitor current 312 flowing from voltage source 302 to ground electrode 108 via wire 112. The calibration system 104 further leverages current meter 318 to monitor current 312 flowing from voltage source 302 to ground electrode 108 via wire 116 and leverages current meter 320 to monitor current 314 flowing from voltage source 302 to ground electrode 108 via wire 120.

A determination is then made as to whether an additional reference bath is included in the calibration process (block 604). The calibration system 104, for instance, determines whether the calibration process involves placement of the ground electrode 108, the wire 112, the wire 116, and the wire 120 in more than one reference bath.

In response to a “Yes” determination at block 604 (e.g., in response to determining that the calibration evaluation process involves moving the calibration board 106 from reference bath 126 to reference bath 128, to reference bath 130, then to reference bath 132), the at least two analyte monitoring devices and the common ground element are moved to an additional reference bath (block 606). The calibration system 104, for instance, moves the ground electrode 108, the wire 112, the wire 116, and the wire 120 from reference bath 126 to reference bath 128. Operation of the procedure 600 then returns to block 602, where currents flowing through each of the analyte monitoring devices are monitored for the additional reference bath.

Alternatively, in response to determining that no additional reference baths are involved in the calibration evaluation process (e.g., in response to determining that the calibration board 106 has been placed in each of reference bath 126, reference bath 128, reference bath 130, and reference bath 132), calibration results are generated for the at least two analyte monitoring devices based on the measured current(s) (block 608). The calibration system 104, for instance, generates calibration results 124. In implementations, the calibration results 124 are representative of a binary indicator, for each analyte monitoring device 122 connected to calibration board 106, as to whether the analyte monitoring device 122 generated signals that accurately reflected known analyte concentrations in each reference bath involved in the calibration evaluation process.

Alternatively or additionally, the calibration results 124 include one or more parameters corresponding to a determined sensitivity of each analyte monitoring device based on the measured current(s) and the known analyte concentrations in each reference bath. For example, the one or more parameters corresponding to a determined sensitivity may be used for converting signals from the analyte sensor to reportable glucose concentrations to a user when the analyte sensor (or an associated sensor with the analyte sensor) is implanted within the user. Alternatively or additionally, the calibration results 124 include detailed information describing data points observed during the calibration evaluation process, such as timestamped metrics indicating currents observed in each analyte monitoring device 122, environmental conditions of the calibration evaluation process, combinations thereof, and so forth.

Having described example procedures in accordance with one or more implementations, consider now an example of a system and device that can be utilized to implement the various techniques described herein.

### Example System and Device

FIG. 7 illustrates an example of a system 700 that includes an example of a computing device 702, which is representative of one or more computing systems and/or devices that may implement the various techniques described herein. This is illustrated through inclusion of the calibration system 104 in the computing device 702. The computing device 702 may be, for example, a server of a service provider, a device associated with a client (e.g., a client device), an on-chip system, and/or any other suitable computing device or computing system.

The example computing device 702 as illustrated includes a processing system 704, one or more computer-readable media 706, and one or more I/O interfaces 708 that are communicatively coupled, one to another. Although not shown, the computing device 702 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 704 is representative of functionality to perform one or more operations using hardware. Accordingly, the processing system 704 is illustrated as including hardware elements 710 that may be configured as processors, functional blocks, and so forth. This may include implementation in hardware as an application specific integrated circuit or other logic device formed using one or more semiconductors. The hardware elements 710 are not limited by the materials from which they are formed, or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable media 706 is illustrated as including memory/storage 712. The memory/storage 712 represents memory/storage capacity associated with one or more computer-readable media. The memory/storage 712 may include volatile media (such as random-access memory (RAM)) and/or nonvolatile media (such as read only memory (ROM), Flash memory, optical disks, magnetic disks, and so forth). The memory/storage 712 may include fixed media (e.g., RAM, ROM, a fixed hard drive, and so on) as well as removable media (e.g., Flash memory, a removable hard drive, an optical disc, and so forth). The computer-readable media 706 may be configured in a variety of other ways as further described below.

Input/output interface(s) 708 are representative of functionality to allow a user to enter commands and information to computing device 702, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 702 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 702. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 702, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 710 and computer-readable media 706 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 710. The computing device 702 may be configured to implement particular instructions and/or functions corresponding to the software and/or hardware modules. Accordingly, implementation of a module that is executable by the computing device 702 as software may be achieved at least partially in hardware, e.g., through use of computer-readable storage media and/or hardware elements 710 of the processing system 704. The instructions and/or functions may be executable/operable by one or more articles of manufacture (for example, one or more computing devices 702 and/or processing systems 704) to implement techniques, modules, and examples described herein.

The techniques described herein may be supported by various configurations of the computing device 702 and are not limited to the specific examples of the techniques described herein. This functionality may also be implemented all or in part through use of a distributed system, such as over a “cloud” 714 via a platform 716 as described below.

The cloud 714 includes and/or is representative of a platform 716 for resources 718. The platform 716 abstracts underlying functionality of hardware (e.g., servers) and software resources of the cloud 714. The resources 718 may include applications and/or data that can be utilized while computer processing is executed on servers that are remote from the computing device 702. Resources 718 can also include services provided over the Internet and/or through a subscriber network, such as a cellular or Wi-Fi network.

The platform 716 may abstract resources and functions to connect the computing device 702 with other computing devices. The platform 716 may also serve to abstract scaling of resources to provide a corresponding level of scale to encountered demand for the resources 718 that are implemented via the platform 716. Accordingly, in an interconnected device embodiment, implementation of functionality described herein may be distributed throughout the system 700. For example, the functionality may be implemented in part on the computing device 702 as well as via the platform 716 that abstracts the functionality of the cloud 714.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. An apparatus configured to calibrate multiple analyte monitoring devices, the apparatus comprising:
a voltage source;
a plurality of electrodes configured to receive an input voltage from the voltage source, each of the plurality of electrodes being connected to a different analyte monitoring device of the multiple analyte monitoring devices; and
a single ground electrode configured to complete a circuit for each of the multiple analyte monitoring devices during calibration of the multiple analyte monitoring devices.

2. The apparatus of claim 1, wherein the single ground electrode is not physically connected to the multiple analyte monitoring devices.

3. The apparatus of claim 1, wherein each of the plurality of electrodes is connected to an analyte sensor of a respective one of the multiple analyte monitoring devices via a contact that conducts the input voltage from the voltage source to an analyte-sensing element of the analyte sensor.

4. The apparatus of claim 3, wherein the analyte-sensing element is inserted subcutaneously into a user when the respective one of the multiple analyte monitoring devices is worn by the user.

5. The apparatus of claim 3, wherein at least one of the plurality of electrodes is connected to an analyte sensor that is used to generate at least one calibration parameter for calibrating one or more different analyte sensors.

6. The apparatus of claim 1, further comprising a plurality of current meters, wherein each of the plurality of current meters monitors a current of the circuit for a corresponding one of the multiple analyte monitoring devices during calibration of the multiple analyte monitoring devices.

7. The apparatus of claim 1, wherein the apparatus is configured such that inserting the single ground electrode into a reference bath causes an analyte-sensing element for each of the multiple analyte monitoring devices to be inserted into the reference bath during calibration of the multiple analyte monitoring devices.

8. The apparatus of claim 7, wherein the reference bath includes a known concentration of an analyte to be measured by the multiple analyte monitoring devices.

9. The apparatus of claim 1, wherein the input voltage from the voltage source causes each of the multiple analyte monitoring devices to generate an electrical current when an analyte-sensing element contacts a fluid that contains an analyte, wherein the electrical current indicates a concentration of the analyte within the fluid.

10. The apparatus of claim 9, wherein the analyte-sensing element includes an enzyme that generates an electrochemical reaction when contacting the analyte, wherein the electrical current is generated from the electrochemical reaction.

11. A system configured to calibrate multiple analyte monitoring devices, the system comprising:
a reference bath containing a solution having a known analyte concentration;
a calibration board comprising:
a plurality of electrodes, each of the plurality of electrodes being connected to a different analyte monitoring device of a plurality of analyte monitoring devices; and
a single ground electrode that contacts the solution of the reference bath;
a voltage source that applies voltage to the plurality of electrodes; and
a plurality of current meters, each of the plurality of current meters monitoring a current flowing from a respective one of the plurality of electrodes to the single ground electrode via a respective one of the plurality of analyte monitoring devices.

12. The system of claim 11, wherein the single ground electrode is not physically connected to the plurality of analyte monitoring devices.

13. The system of claim 11, wherein the voltage source applies a constant voltage to the plurality of electrodes while the single ground electrode and an analyte-sensing element of each of the plurality of analyte monitoring devices contacts the solution of the reference bath.

14. The system of claim 11, wherein the voltage source causes each of the plurality of analyte monitoring devices to generate the current flowing from the respective one of the plurality of electrodes to the single ground electrode, wherein the current indicates an observed analyte concentration of the solution of the reference bath.

15. The system of claim 11, wherein each of the plurality of analyte monitoring devices includes an analyte-sensing element comprising an enzyme that generates an electrochemical reaction when contacting the known analyte concentration in the solution of the reference bath, wherein the current is generated from the electrochemical reaction.

16. The system of claim 11, wherein the plurality of analyte monitoring devices are continuous glucose monitoring devices and the solution of the reference bath comprises a known concentration of glucose.

17. A method comprising:
applying a voltage to an analyte monitoring device connected to a calibration board while a sensor of the analyte monitoring device and a single ground electrode of the calibration board are in contact with a reference bath;
measuring, for the analyte monitoring device, a current that flows through the sensor to the single ground electrode as a result of applying the voltage; and
generating calibration results for the analyte monitoring device based on the current.

18. The method of claim 17, wherein the single ground electrode of the calibration board is not physically connected to the analyte monitoring device.

19. The method of claim 17, wherein the current indicates an analyte concentration of a solution contained in the reference bath that is observed by the analyte monitoring device.

20. The method of claim 17, wherein the sensor of the analyte monitoring device includes an enzyme that generates an electrochemical reaction when contacting an analyte included in a solution contained within the reference bath, wherein the current is generated by the electrochemical reaction.
